(19) United States
(12) Patent Application Publication
(10) Pub. No.: US 2022/0011257 A1
(43) Pub. Date: Jan. 13, 2022

(54) GAS SENSOR

(71) Applicant: DENSO CORPORATION, Kariya-city (JP)

(72) Inventors: Toshihiko HARADA, Nisshin-city (JP); Keigo MIZUTANI, Nisshin-city (JP); Hironobu SHIMOKAWA, Nisshin-city (JP); Yusuke KAWAMOTO, Kariya-city (JP); Hiroki ICHIKAWA, Kariya-city (JP)

(21) Appl. No.: 17/485,962

(22) Filed: Sep. 27, 2021

Related U.S. Application Data
(63) Continuation of application No. PCT/JP2020/002489, filed on Jan. 24, 2020.

(30) Foreign Application Priority Data
Mar. 28, 2019 (JP) 2019-063493

(51) Int. Cl. G01N 27/409; G01N 27/41; G01N 27/407

(52) U.S. Cl. CPC G01N 27/409; G01N 27/4077; G01N 27/4071; G01N 27/41

(57) ABSTRACT

A sensor element of a gas sensor includes a solid electrolyte body, a first insulator, a gas chamber, a second insulator, a reference gas duct, a pump electrode, a sensor electrode, a reference electrode, and a shield layer. The shield layer is formed of an insulating ceramic material and covers a sensor-side electrode portion of the reference electrode, the sensor-side electrode portion being arranged to overlap the sensor electrode through the solid electrolyte body.

### CROSS-REFERENCE TO RELATED APPLICATION

The present application is a continuation application of International Application No. PCT/JP2020/002489, filed on Jan. 24, 2020, which claims priority to Japanese Patent Application No. 2019-063493, filed on Mar. 28, 2019. The contents of these applications are incorporated herein by reference in their entirety.

### BACKGROUND

### Technical Field

The present invention relates to a gas sensor including a sensor element for detecting the concentration of a specific gas component in a detection target gas.

### Background Art

In the exhaust pipe of an internal combustion engine in a vehicle, a gas sensor is used to detect the concentration of a specific gas component such as nitrogen oxides (NOx) in an exhaust gas flowing in the exhaust pipe which is the detection target gas. For example, the gas sensor detects the concentration of the specific gas component flowing out of a catalyst arranged in the exhaust pipe and monitor whether the catalyst is normally functioning.

### SUMMARY

In the present disclosure, provided is a gas sensor as the following.

The gas sensor includes a sensor element for detecting the concentration of a specific gas component, the sensor element includes: a solid electrolyte body; a first insulator that is laminated on the first surface of the solid electrolyte body; a gas chamber that is defined by the first surface and a recess of the first insulator; a second insulator that is laminated on the second surface of the solid electrolyte body; a reference gas duct that is defined by the second surface and a groove of the second insulator; a pump electrode that is provided on the first surface and housed in the gas chamber; a sensor electrode that is provided on the first surface and housed in the gas chamber; a reference electrode arranged on the second surface of the solid electrode to overlap both the pump electrode and the sensor electrode through the solid electrolyte body; and an insulating shield layer arranged to cover a portion of the reference electrode in a contact state or non-contact state with the portion of the reference electrode, the portion of the reference electrode being arranged to overlap the sensor electrode through the solid electrolyte body.

### BRIEF DESCRIPTION OF THE DRAWINGS

Objectives, features, and advantages of the present disclosure will be become more clearly by the detailed description below with reference to the attached drawings. The drawings of the present disclosure are as follows.

FIG. 1 is a cross-sectional view of a gas sensor according to a first embodiment.

FIG. 2 is a cross-sectional view of a sensor element according to the first embodiment.

FIG. 3 is a cross-sectional view of the sensor element taken along line of FIG. 2 according to the first embodiment.

FIG. 4 is a cross-sectional view of the sensor element taken along line IV-IV of FIG. 2 according to the first embodiment.

FIG. 5 is a cross-sectional view of the sensor element taken along line V-V of FIG. 2 according to the first embodiment.

FIG. 6 is a cross-sectional view of the sensor element taken along line VI-VI of FIG. 2 according to the first embodiment.

FIG. 7 is a VI-VI cross-sectional equivalent view of FIG. 2 showing another sensor element according to the first embodiment.

FIG. 8 is a VI-VI cross-sectional equivalent view of FIG. 2 showing further another sensor element according to the first embodiment.

FIG. 9 is a cross-sectional view of a sensor element according to a second embodiment.

FIG. 10 is a cross-sectional view of the sensor element taken along line X-X of FIG. 9 according to the second embodiment.

FIG. 11 is a X-X cross-sectional equivalent view of FIG. 9 showing another sensor element according to the second embodiment.

FIG. 12 is a cross-sectional view of a sensor element according to a third embodiment.

FIG. 13 is a cross-sectional view of the sensor element taken along line XIII-XIII of FIG. 12 according to the third embodiment.

FIG. 14 is a cross-sectional view of the sensor element taken along line XIV-XIV of FIG. 12 according to the third embodiment.

FIG. 15 is a cross-sectional view of the sensor element taken along line XIII-XIII of FIG. 12 according to the third embodiment.

FIG. 16 is a XIV-XIV cross-sectional equivalent view of FIG. 12 showing another sensor element according to the third embodiment.

FIG. 17 is a IV-IV cross-sectional equivalent view of FIG. 2 showing a sensor element according to a fourth embodiment.

FIG. 18 is a V-V cross-sectional equivalent view of FIG. 2 showing the sensor element according to the fourth embodiment.

FIG. 19 is a IV-IV cross-sectional equivalent view of FIG. 2 showing another sensor element according to the fourth embodiment.

FIG. 20 is a VI-VI cross-sectional equivalent view of FIG. 2 showing another sensor element according to the fourth embodiment.

### DETAILED DESCRIPTION OF THE PREFERRED EMBODIMENTS

Such a gas sensor detecting the concentration of the specific gas component includes, for example, a solid electrolyte body, a gas chamber that is formed in an insulator laminated on the first surface of the solid electrolyte body, and a reference gas duct that is formed in an insulator laminated on the second surface of the solid electrolyte body. A pump electrode and a sensor electrode housed in the gas chamber are provided on the first surface of the solid electrolyte body, and a reference electrode housed in the reference gas duct is provided on the second surface of the solid electrolyte body. The pump electrode has catalytic activity against oxygen, and the sensor electrode has catalytic activity against oxygen and the specific gas component.

When a direct-current voltage is applied between the pump electrode and the reference electrode, oxygen is pumped out of the detection target gas in the gas chamber. In addition, the concentration of the specific gas component in the detection target gas within the gas chamber is detected by the electric current generated between the sensor electrode and the reference electrode between which the direct-current voltage is applied. Such a gas sensor is described in PTL 1, for example.

In the gas sensor of PTL 1, the monitor electrode having catalytic activity against oxygen is provided alongside the sensor electrode on the first surface of the solid electrolyte body. The concentration of the specific gas component in the detection target gas within the gas chamber is detected by the electric current generated between the monitor electrode and the reference electrode between which a direct-current voltage is applied. By subtracting the electric current generated between the monitor electrode and the reference electrode from the electric current generated between the sensor electrode and the reference electrode, it is possible to alleviate the influence of the residual oxygen in the detection target gas on the concentration of the specific gas component.

[PTL 1] JP 2017-40660 A

In regard to the gas sensor arranged and used in the exhaust pipe of the internal combustion engine in a vehicle, when the operating state of the internal combustion engine becomes a fuel-cut state where the injection of a fuel is stopped, the concentration of oxygen in the detection target gas sharply increases. In this case, a large amount of oxygen is pumped from the pump electrode to the reference electrode via the solid electrolyte body by the direct-current voltage applied between the pump electrode and the reference electrode. At this time, the oxygen concentration in the atmosphere that is a reference gas within the reference gas duct increases temporarily. Under the influence of this increase, the electric current generated between the sensor electrode and the reference electrode increases temporarily.

Thus, the value of the electric current generated between the sensor electrode and the reference electrode may become larger even though actually the sensor electrode has not detected the specific gas component. This may cause an error in the detection of the concentration of the specific gas component. On the other hand, in the gas sensor of PTL 1, the sensor electrode and the monitor electrode are affected by the temporary increase of the oxygen concentration in the atmosphere within the reference gas duct. This possibly alleviates the error occurring in the detection of the concentration of the specific gas component.

However, since the sensor electrode and the monitor electrode are different in composition, the sensor electrode and the monitor electrode are also different in electrostatic capacitance. Thus, the temporal increase of the oxygen concentration in the atmosphere within the reference gas duct produces a difference between the electric current generated between the sensor electrode and the reference electrode and the electric current generated between the monitor electrode and the reference electrode. Accordingly, it is difficult to eliminate the error occurring in the detection of the concentration of the specific gas component.

The present disclosure is obtained to provide a gas sensor that suppresses an error in the detection of the concentration of the specific gas component even with a sharp increase in the oxygen concentration in the reference gas.

An aspect of the present disclosure is a gas sensor that includes a sensor element for detecting the concentration of a specific gas component in a detection target gas, wherein the sensor element includes: a solid electrolyte body that has ionic conductivity, and has a first surface and a second surface; a first insulator that is laminated on the first surface of the solid electrolyte body and has a recess; a gas chamber into which the detection target gas is introduced, the gas chamber being defined by the first surface of the solid electrolyte body and the recess of the first insulator; a second insulator that is laminated on the second surface of the solid electrolyte body, and has a groove; a reference gas duct into which a reference gas is introduced, the reference gas duct being defined by the second surface of the solid electrolyte body and the groove of the second insulator; a pump electrode for adjusting the concentration of oxygen in the detection target gas, the pump electrode being provided on the first surface of the solid electrolyte body and housed in the gas chamber; a sensor electrode for detecting the concentration of the specific gas component in the detection target gas that includes the oxygen whose concentration has been adjusted by the pump electrode, the sensor electrode being provided on the first surface of the solid electrolyte body and housed in the gas chamber; a reference electrode arranged on the second surface of the solid electrode to overlap both the pump electrode and the sensor electrode through the solid electrolyte body; and an insulating shield layer arranged to cover a portion of the reference electrode in a contact state or non-contact state with the portion of the reference electrode, the portion of the reference electrode being arranged to overlap the sensor electrode through the solid electrolyte body.

In the gas sensor of the above aspect, the insulating shield layer is arranged to cover a portion of the reference electrode being arranged to overlap the sensor electrode through the solid electrolyte body. The portion of the reference electrode being arranged to overlap the sensor electrode through the solid electrolyte body refers to the portion of the reference electrode facing the sensor electrode through the solid electrolyte body. When the specific gas component is detected, the electric current flows between the sensor electrode and the portion of the reference electrode being arranged to overlap the sensor electrode through the solid electrolyte body.

When the oxygen concentration in the detection target gas sharply changes because the operating state of the internal combustion engine becomes the fuel-cut state or the like, a large amount of oxygen is pumped from the pump electrode to the reference electrode by the actions of the pump electrode and the reference electrode. This leads to a sharp increase of the oxygen concentration in the reference gas such as the atmosphere within the reference gas duct.

However, the portion of the reference electrode overlapping the sensor electrode through the solid electrolyte body is covered with the shield layer. Thus, even if the oxygen concentration in the reference gas within the reference gas duct sharply increases, the oxygen concentration in the reference gas in contact with the portion of the reference electrode overlapping the sensor electrode through the solid electrolyte body will not sharply increase. Accordingly, it is possible to prevent the electric current generated between the sensor electrode and the portion of the reference electrode overlapping the sensor electrode through the solid electrolyte body from being affected by the sharp increase of the oxygen concentration in the reference gas.

Therefore, according to the gas sensor of the above aspect, even with a sharp increase of the oxygen concentration in the reference gas, it is possible that there is almost no error in the detection of the concentration of the specific gas component.

The reference signs in parentheses for the components described in relation to the aspect of the present disclosure indicate the correspondence with the reference signs for the components of the embodiments illustrated in the drawings, but are not intended to limit the components to the contents of the embodiments.

Preferred embodiments of the above gas sensor will be described with reference to the drawings.

### First Embodiment

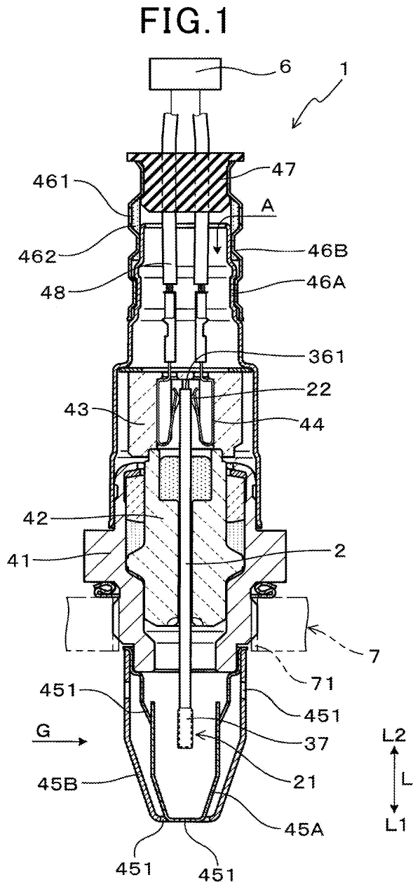

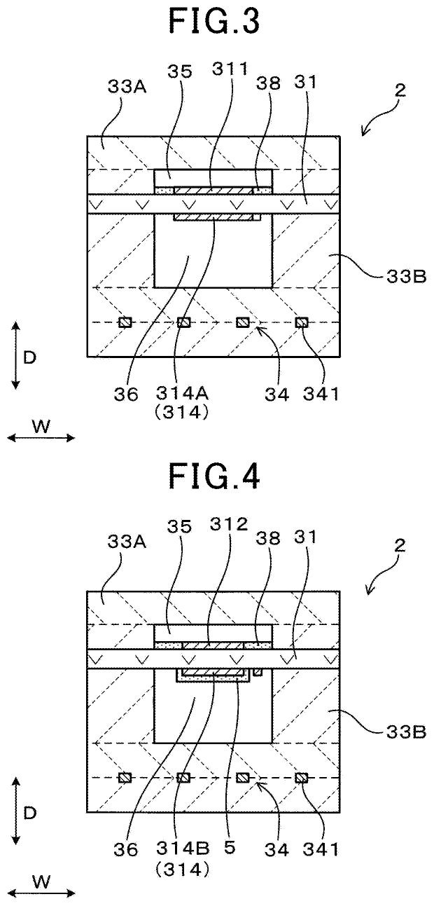

A gas sensor 1 of the present embodiment includes a sensor element 2 for detecting the concentration of a specific gas component in a detection target gas G as illustrated in FIGS. 1 to 6. The sensor element 2 has a solid electrolyte body 31, a first insulator 33A, a gas chamber 35, a second insulator 33B, a reference gas duct 36, a pump electrode 311, a sensor electrode 312, a reference electrode 314, and a shield layer 5.

The solid electrolyte body 31 has ionic conductivity by which oxygen ions are conducted at a predetermined activation temperature. The first insulator 33A is laminated on a first surface 301 of the solid electrolyte body 31. The gas chamber 35 is defined by the first surface 301 of the solid electrolyte body 31 and a recess formed in the first insulator 33A, thereby producing a space into which the detection target gas G is introduced. The second insulator 33B is laminated on a second surface 302 of the solid electrolyte body 31. The reference gas duct 36 is defined by the second surface 302 of the solid electrolyte body 31 and a groove formed in the second insulator 33B, thereby producing a flow path through which a reference gas A is introduced.

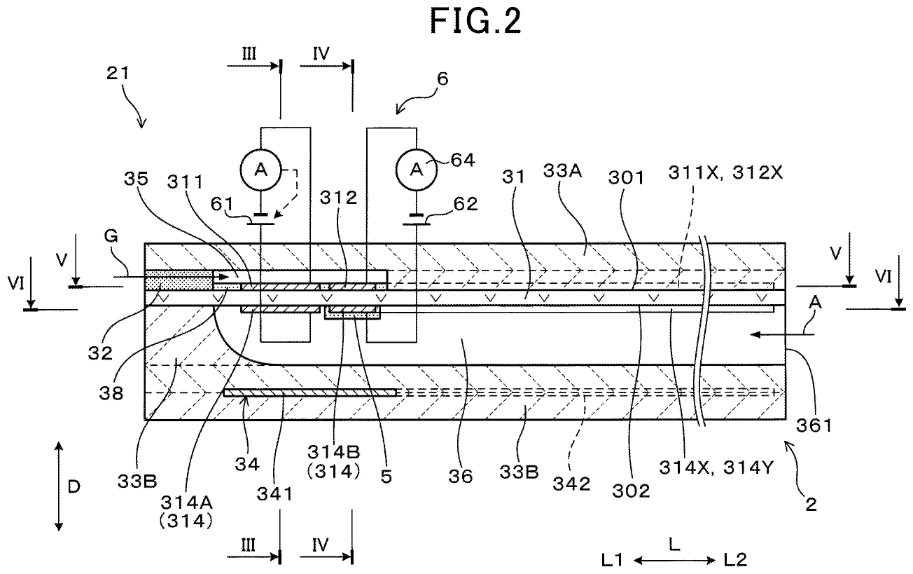

As illustrated in FIGS. 2, 3, and 5, the pump electrode 311 is provided on the first surface 301 of the solid electrolyte body 31 and is housed in the gas chamber 35. The pump electrode 311 is used to adjust the concentration of oxygen in the detection target gas G The sensor electrode 312 is provided on the first surface 301 of the solid electrolyte body 31 and is housed in the gas chamber 35. The sensor electrode 312 is used to detect the concentration of a specific gas component in the detection target gas G after the adjustment of the concentration of oxygen by the pump electrode 311.

As illustrated in FIGS. 2, 4, and 6, the reference electrode 314 is provided on the second surface 302 of the solid electrolyte body 31 opposite to the first surface 301, to cover the pump electrode 311 and the sensor electrode 312 through the solid electrolyte body 31. The shield layer 5 is made of an insulating ceramic material and covers a sensor-side electrode portion 314B that is a portion of the reference electrode 314 overlapping the sensor electrode 312 through the solid electrolyte body 31.

Hereinafter, the gas sensor 1 of the present embodiment will be described in detail.

### (Gas Sensor 1)

As illustrated in FIG. 1, the gas sensor 1 is arranged at an attachment port 71 of an exhaust pipe 7 of internal combustion engine (engine) in a vehicle and is used to detect the concentration of a nitrogen oxide (NOx) as a specific gas component in the detection target gas G that is an exhaust gas flowing through the exhaust pipe 7. The NOx includes nitrogen monoxide (NO), nitrogen dioxide (NO₂), and others.

A catalyst is arranged in the exhaust pipe 7 to purify harmful substances in the detection target gas G The gas sensor 1 can be arranged, for example, at a position downstream of the catalyst in the flow direction of the detection target gas G in the exhaust pipe 7.

### (Sensor Element 2)

As illustrated in FIGS. 2 to 6, the sensor element 2 of the present embodiment is formed in the shape of a rectangular parallelepiped. The sensor element 2 is of a laminated type in which the first insulator 33A, the second insulator 33B, and the like are laminated on the solid electrolyte body 31. The sensor element 2 has, on a front end side L1 of a longitudinal direction L, a detection unit 21 formed by the pump electrode 311, the sensor electrode 312, the reference electrode 314, and a portion of the solid electrolyte body 31 sandwiched between the pump electrode 311, the sensor electrode 312, and the reference electrode 314.

In the present embodiment, the longitudinal direction L of the sensor element 2 refers to the direction in which the sensor element 2 extends along the longitudinal side. The direction orthogonal to the longitudinal direction L and in which the solid electrolyte body 31 and the insulators 33A and 33B are laminated to one another, in other words, the direction in which the solid electrolyte body 31, the insulators 33A and 33B, and a heating element 34 are laminated to one another, will be called lamination direction D. The direction orthogonal to the longitudinal direction L and the lamination direction D will be called width direction W. The side of the sensor element 2 exposed to the detection target gas G along the longitudinal direction L will be called front end side L1, and the side opposite to the front end side L1 will be called rearend side L2.

### (Solid Electrolyte Body 31, Pump Electrode 311, Sensor Electrode 312, and Reference Electrode 314)

As illustrated in FIGS. 2 to 6, the solid electrolyte body 31 has a plate-like shape and possesses oxygen ion (O²⁻) conductivity at a predetermined activation temperature. The solid electrolyte body 31 is formed of a zirconia-based oxide and contains zirconia as the main component (50% or more by mass). The solid electrolyte body 31 includes stabilized zirconia or partially stabilized zirconia in which the zirconia is partially substituted by a rare earth metal element or an alkali earth metal element. The zirconia constituting the solid electrolyte body 31 can be partially substituted by yttria, scandia, or calcia.

The pump electrode 311 contains platinum as a noble metal having catalytic activity against oxygen and a zirconia-based oxide as a material in common with the solid electrolyte body 31. The common material is used to, when a paste of electrode material is printed (applied) on the solid electrolyte body 31 and the two are fired, maintain the strength of bonding between the pump electrode 311 and the like formed by the electrode material and the solid electrolyte body 31.

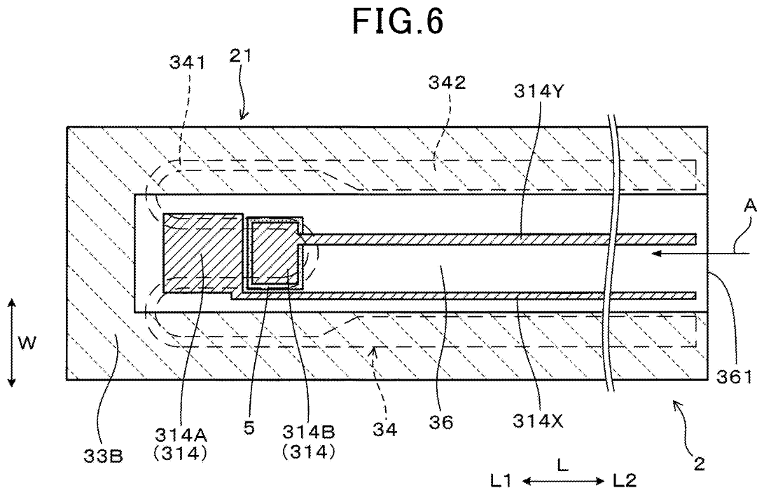

The sensor electrode 312 contains a noble metal having catalytic activity against oxygen and NOx as a specific gas component and a zirconia oxide as a material in common with the solid electrolyte body 31. The sensor electrode 312 contains platinum having catalytic activity against oxygen and rhodium having catalytic activity against NOx. The sensor electrode 312 can use, besides rhodium, a noble metal having catalytic activity against NOx. The reference electrode 314 contains platinum as a noble metal having catalytic activity against oxygen and a zirconia-based oxide as a material in common with the solid electrolyte body 31. As illustrated in FIGS. 2 and 6, the reference electrode 314 of the present embodiment is divided into a pump-side electrode portion 314A that overlaps the pump electrode 311 in the lamination direction D through the solid electrolyte body 31, and a sensor-side electrode portion 314B that overlaps the sensor electrode 312 in the lamination direction D through the solid electrolyte body 31. The pump-side electrode portion 314A has the same area as that of the pump electrode 311 and is arranged at a position facing the pump electrode 311 through the solid electrolyte body 31. The sensor-side electrode portion 314B has the same area as that of the sensor electrode 312 and is arranged at a position facing the sensor electrode 312 through the solid electrolyte body 31. The “overlapping in the lamination direction D through the solid electrolyte body 31” means presenting at an overlapping position as viewed in the lamination direction D.

By separating the reference electrode 314 into the pump-side electrode portion 314A and the sensor-side electrode portion 314B, the sensor-side electrode portion 314B can be covered alone with the shield layer 5. Thus, the portion of the reference electrode 314 affecting the detection of the specific gas component such as NOx can be effectively covered.

The pump electrode 311 is arranged on the first surface 301 of the solid electrolyte body 31, at a position of the front end side L1 of the gas chamber 35 in the longitudinal direction L. The pump electrode 311 is arranged on the first surface 301 of the solid electrolyte body 31, at a position adjacent to the rear end side L2 of a diffusion resistance layer 32 described later in the longitudinal direction L. The sensor electrode 312 is arranged on the first surface 301 of the solid electrolyte body 31, at a position adjacent to the front end side L1 of the pump electrode 311 in the longitudinal direction L. The detection target gas G introduced from the diffusion resistance layer 32 into the gas chamber 35 contacts the pump electrode 311 to decrease the concentration of oxygen, and then comes into contact with the sensor electrode 312.

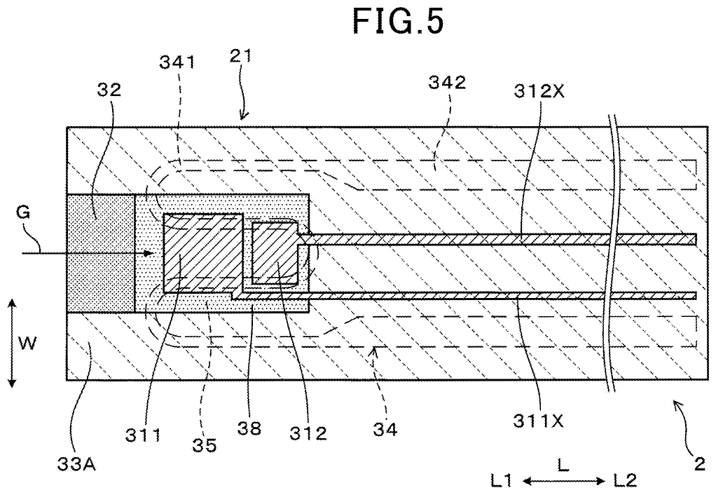

As illustrated in FIG. 5, the pump electrode 311 is connected to a pump electrode lead portion 311X that is provided on the first surface 301 of the solid electrolyte body 31 for electrical connection the pump electrode 311 to the outside of the gas sensor 1. The pump electrode lead portion 311X is formed from the rear end position of the pump electrode 311 along the longitudinal direction L to the rear end portion of the solid electrolyte body 31 along the longitudinal direction L.

The sensor electrode 312 is connected to a sensor electrode lead portion 312X that is provided on the first surface 301 of the solid electrolyte body 31 for electrical connection the sensor electrode 312 to the outside of the gas sensor 1. The sensor electrode lead portion 312X is formed from the rear end position of the sensor electrode 312 along the longitudinal direction L to the rear end portion of the solid electrolyte body 31 along the longitudinal direction L.

As illustrated in FIG. 6, the pump-side electrode portion 314A of the reference electrode 314 is connected to a first reference electrode lead portion 314X that is provided on the second surface 302 of the solid electrolyte body 31 for electrically connecting the pump-side electrode portion 314A to the outside of the gas sensor 1. The first reference electrode lead portion 314X is formed from the rear end position of the pump-side electrode portion 314A along the longitudinal direction L to the rear end portion of the solid electrolyte body 31 along the longitudinal direction L.

The sensor-side electrode portion 314B of the reference electrode 314 is connected to a second reference electrode lead portion 314Y that is provided on the second surface 302 of the solid electrolyte body 31 for electrically connecting the sensor-side electrode portion 314B to the outside of the gas sensor 1. The second reference electrode lead portion 314Y is formed from the rear end position of the sensor-side electrode portion 314B along the longitudinal direction L to the rear end portion of the solid electrolyte body 31 along the longitudinal direction L.

The pump electrode lead portion 311X, the sensor electrode lead portion 312X, the first reference electrode lead portion 314X, and the second reference electrode lead portion 314Y are mostly formed along the longitudinal direction L. As illustrated in FIG. 1, the respective rear end portions of the pump electrode lead portion 311X, the sensor electrode lead portion 312X, the first reference electrode lead portion 314X, and the second reference electrode lead portion 314Y are made electrically continuous with corresponding contact parts 22 provided on the surface of the sensor element 2 via a through hole in the first insulator 33A or the second insulator 33B. Contact terminals 44 of lead wires 48 wired to a sensor control unit 6 outside the gas sensor 1 are in contact with the contact parts 22 of the sensor element 2.

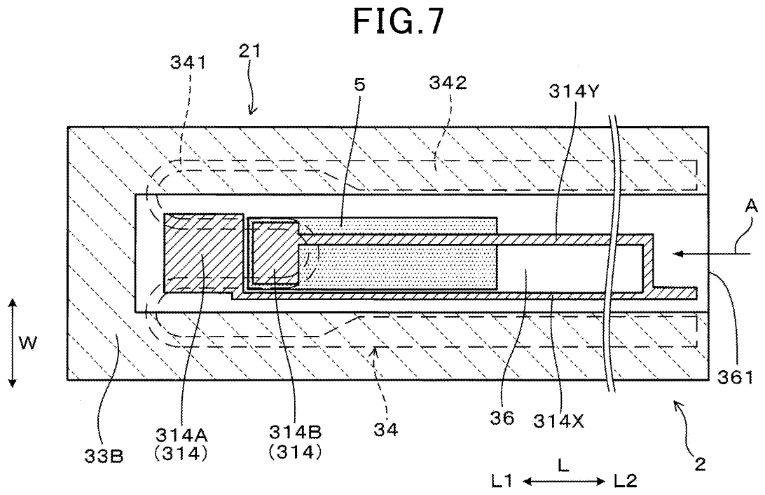

As illustrated in FIG. 7, the rear end portion of the first reference electrode lead portion 314X and the rear end portion of the second reference electrode lead portion 314Y may be connected together on the second surface 302 of the solid electrolyte body 31. In this case, even if the reference electrode 314 is separated (divided) into a plurality of pieces, the contact parts 22 can be unified into one to reduce the number of the contact terminals 44 and lead wires 48 used.

As illustrated in FIG. 6, since the sensor-side electrode portion 314B of the reference electrode 314 is covered with the shield layer 5, the oxygen in the reference gas A is supplied from the second reference electrode lead portion 314Y to the sensor-side electrode portion 314B. Thus, in order to easily take the oxygen from the reference gas A into the second reference electrode lead portion 314Y, the area of the cross section of the second reference electrode lead portion 314Y orthogonal to the longitudinal direction L can be made larger than the area of the cross section of the pump electrode lead portion 311X orthogonal to the longitudinal direction L, the area of the cross section of the sensor electrode lead portion 312X orthogonal to the longitudinal direction L, and the area of the cross section of the first reference electrode lead portion 314X orthogonal to the longitudinal direction L.

The area of the cross section of the second reference electrode lead portion 314Y orthogonal to the longitudinal direction L can be made larger than the areas of the cross sections of the pump electrode lead portion 311X, the sensor electrode lead portion 312X, and the first reference electrode lead portion 314X orthogonal to the longitudinal direction L, by increasing at least one of the thickness along the lamination direction D and the width along the width direction W.

The area of the cross section of the second reference electrode lead portion 314Y orthogonal to the longitudinal direction L can be within the range of 0.01 to 0.1 mm². The thickness of the second reference electrode lead portion 314Y along the lamination direction D can be within the range of 10 to 50 μm. The width of the second reference electrode lead portion 314Y along the width direction W can be within the range of 1.0 to 2.0 mm.

The areas of the cross sections of the pump electrode lead portion 311X, the sensor electrode lead portion 312X, and the first reference electrode lead portion 314X orthogonal to the longitudinal direction L can be within the range of 0.0015 to 0.01 mm². The thicknesses of the pump electrode lead portion 311X, the sensor electrode lead portion 312X, and the first reference electrode lead portion 314X along the lamination direction D can be within the range of 5 to 10 μm. The widths of the pump electrode lead portion 311X, the sensor electrode lead portion 312X, and the first reference electrode lead portion 314X along the width direction W can be within the range of 0.3 to 1.0 mm

As illustrated in FIGS. 2 to 5, a portion where the gas chamber 35 is formed on the first surface 301 of the solid electrolyte body 31 includes a first portion where the pump electrode 311 and the sensor electrode 312 are provided and a second portion where the pump electrode 311 and the sensor electrode 312 are not provided, and an insulation layer 38 made of an insulating ceramic material such as alumina is provided at the second portion. The insulation layer 38 can be formed to have a thickness equivalent to the thickness of the pump electrode 311 and the sensor electrode 312 along the lamination direction D.

### (Shield Layer 5)

As illustrated in FIGS. 2, 4, and 6, the shield layer 5 of the present embodiment is provided so as not to bury the flow path in the reference gas duct 36 but so as to be in contact with the sensor-side electrode portion 314B and cover the entire sensor-side electrode portion 314B. The shield layer 5 is formed with a uniform thickness on the surface of the sensor-side electrode portion 314B. A portion of the shield layer 5 surrounds the sensor-side electrode portion 314B and is in contact with the second surface 302 of the solid electrolyte body 31.

The shield layer 5 is formed of a dense ceramic material with the property that is difficult for reference gas to transit A. The ceramic material is made of particles of a metallic oxide such as alumina. The “dense” means that there is hardly formed a gap between particles of a metallic oxide, or the gap formed between particles of a metallic oxide is small to the extent that a gas such as the reference gas A cannot be passed through the gap.

Since the entire sensor-side electrode portion 314B is covered with the shield layer 5, it is considered that the oxygen moves from the sensor-side electrode portion 314B to the atmosphere as the reference gas A via the second reference electrode lead portion 314Y. The concentration of oxygen in the atmosphere as the reference gas A in contact with the sensor-side electrode portion 314B is hardly affected by changes in the concentration of oxygen in the reference gas A within the reference gas duct 36.

As illustrated in FIG. 7, the shield layer 5 may continuously cover the entire sensor-side electrode portion 314B and a portion of the second reference electrode lead portion 314Y of the reference electrode 314. The portion of the second reference electrode lead portion 314Y covered with the shield layer 5 can be made longer than the length of the sensor-side electrode portion 314B along the longitudinal direction L. The shield layer 5 can also cover a 20 percent or more of the entire length of the second reference electrode lead portion 314Y along the longitudinal direction L, from the front end side L1 of the longitudinal direction L. In this case, it is possible to suppress the influence of changes in the concentration of oxygen in the reference gas A within the reference gas duct 36 on the second reference electrode lead portion 314Y. The shield layer 5 may cover the entire sensor-side electrode portion 314B and the entire second reference electrode lead portion 314Y.

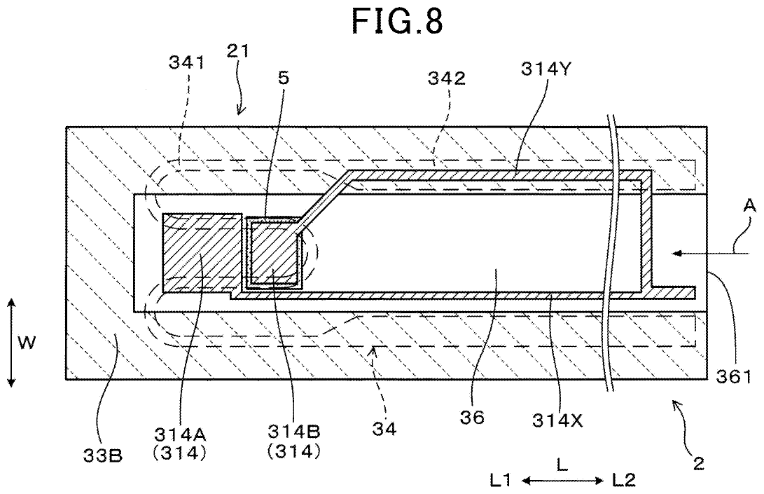

As illustrated in FIG. 8, the second reference electrode lead portion 314Y may be embedded in the boundary between the solid electrolyte body 31 and the second insulator 33B or in the second insulator 33B, except for the portion on the front end side L1 in the longitudinal direction L which is connected to the sensor-side electrode portion 314B. In this case, the shield layer 5 can cover the sensor-side electrode portion 314B and the portion of the second reference electrode lead portion 314Y which is on the front end side L1 in the longitudinal direction L and is connected to the sensor-side electrode portion 314B.

### (Gas Chamber 35)

As illustrated in FIGS. 2 to 5, the gas chamber 35 is formed adjacent to the first surface 301 of the solid electrolyte body 31 and is surrounded by the first insulator 33A and the solid electrolyte body 31. The gas chamber 35 is formed at a portion of the first insulator 33A on the front end side L1 in the longitudinal direction L, at a position where to house the pump electrode 311. The gas chamber 35 is formed as a space closed by the first insulator 33A, the diffusion resistance layer 32, and the solid electrolyte body 31. The detection target gas G flowing in the exhaust pipe 7 passes through the diffusion resistance layer 32 and is introduced into the gas chamber 35.

### (Diffusion Resistance Layer 32)

As illustrated in FIGS. 2 and 5, the diffusion resistance layer 32 is provided at the front end portion of the first insulator 33A along the longitudinal direction L to introduce the detection target gas G into the gas chamber 35 under a predetermined diffusion resistance. The diffusion resistance layer 32 of the present embodiment is adjacent to the front end side L1 of the gas chamber 35 along the longitudinal direction L. The diffusion resistance layer 32 is arranged in an introduction port opened in the first insulator 33A adjacent to the front end side L1 of the gas chamber 35 along the longitudinal direction L. The diffusion resistance layer 32 is formed of a porous metallic oxide such as alumina. The diffusion rate (flow rate) of the detection target gas G introduced into the gas chamber 35 is determined by limiting the speed at which the detection target gas G passes through the air holes in the diffusion resistance layer 32.

The diffusion resistance layer 32 may be adjacent to both sides of the gas chamber 35 along the width direction W. In this case, the diffusion resistance layer 32 is arranged in the introduction port of the first insulator 33A adjacent to both sides of the gas chamber 35 along the width direction W. Also in this case, the diffusion resistance layer 32 can be formed on both sides of the gas chamber 35 along the width direction W, at positions nearer the front end side L1 than the formation position of the pump electrode 311 along the longitudinal direction L. Instead of forming the diffusion resistance layer 32 on the sensor element 2, the first insulator 33A may be provided with a pin hole that is a small through-hole in communication with the gas chamber 35.

The gas sensor 1 of the present embodiment is a limited-current sensor using the gas chamber 35 and the diffusion resistance layer 32. More specifically, the gas sensor 1 detects the concentration of NOx as the specific gas component, taking advantage of a limited-current property that, when a predetermined direct-current voltage is applied between the sensor electrode 312 and the sensor-side electrode portion 314B of the reference electrode 314, the electric current value becomes constant, regardless of changes in voltage, due to diffusion control (inflow limiting) of the detection target gas G by the diffusion resistance layer 32.

### (Reference Gas Duct 36)

As illustrated in FIGS. 2, 3, 4, and 6, the reference gas duct 36 is surrounded by the second insulator 33B and the solid electrolyte body 31 and is formed adjacent to the second surface 302 of the solid electrolyte body 31. The reference gas duct 36 is formed from a portion of the second insulator 33B that houses the reference electrode 314 along the longitudinal direction L to the rear end position exposed to the reference gas A such as the atmosphere, along the longitudinal direction L of the sensor element 2. A rear end opening 361 as a reference gas introduction part is formed in the reference gas duct 36 at the rear end position of the sensor element 2 along the longitudinal direction L. The reference gas duct 36 is formed from the rear end opening 361 to the position where to overlap the gas chamber 35 in the lamination direction D through the solid electrolyte body 31. The reference gas A is introduced into the reference gas duct 36 through the rear end opening 361.

The area of the cross section of the reference gas duct 36 orthogonal to the longitudinal direction L is larger than the area of the cross section of the gas chamber 35 orthogonal to the longitudinal direction L. The thickness (width) of the reference gas duct 36 along the lamination direction D is larger than the thickness (width) of the gas chamber 35 along the lamination direction D. Since the cross-section area, thickness, and volume of the reference gas duct 36 are larger than the cross-section area, thickness, and volume of the gas chamber 35, it is possible to provide a sufficient supply of oxygen in the reference gas A for reacting the unburned gas in the pump electrode 311 from the reference gas duct 36 to the pump electrode 311.

### (Heating Element 34)

As illustrated in FIGS. 2 to 6, the heating element 34 is buried in the second insulator 33B forming the reference gas duct 36. The heating element 34 includes a heating part 341 that generates heat when energized, and a heating element lead portion 342 that is connected to the heating part 341. The heating part 341 is arranged at a position at least partially overlapping the pump electrode 311, the sensor electrode 312, and the reference electrode 314 in the lamination direction D in which the solid electrolyte body 31 and the insulators 33A, 33B are laminated to one another.

The heating element 34 includes the heating part 341 that generates heat when energized and a pair of heating element lead portions 342 that are respectively connected to the rear end sides L2 of the heating part 341 along the longitudinal direction L. The heating part 341 is formed of a linear conductor that meanders with linear portions and curved portions. The linear portions of the heating part 341 of the present embodiment are formed in parallel with the longitudinal direction L. Each of the heating element lead portions 342 is formed of linear conductor. The resistance value of the heating part 341 per unit length is larger than the resistance value of the heating element lead portions 342 per unit length. The heating element lead portion 342 are drawn up to the rear end side L2 along the longitudinal direction L. The heating element 34 contains an electrically conductive metallic material.

As illustrated in FIGS. 5 and 6, the heating part 341 of the present embodiment is formed to meander in the longitudinal direction L at a position on the front end side L1 of the heating element 34 along the longitudinal direction L. The heating part 341 may be formed to meander in the width direction W. The heating part 341 is arranged at a position facing the pump electrode 311, the sensor electrode 312, and the reference electrode 314 in the lamination direction D orthogonal to the longitudinal direction L. In other words, the heating part 341 is arranged on the front end side L1 of the sensor element 2 along the longitudinal direction L, to cover the pump electrode 311, the sensor electrode 312, and the reference electrode 314 in the lamination direction D.

The cross-section area of the heating part 341 is smaller than the cross-section area of each of the heating element lead portions 342, and the resistance value of the heating part 341 per unit length is higher than the resistance value of each of the heating element lead portions 342 per unit length. The cross-section area here refers to the area of cross section of the surface orthogonal to the direction in which the heating part 341 and the heating element lead portions 342 extend. When a voltage is applied to the pair of heating element lead portions 342, the heating part 341 generates heat due to Joule heat, thereby heating the detection unit 21 and the periphery of the detection unit 21.

When the heating part 341 generates heat with Energization of the heating element lead portions 342, the sensor electrode 312, the sensor electrode 312, the reference electrode 314, and the portion of solid electrolyte body 31 sandwiched between the electrodes 311, 312, and 314 are heated to the target temperature.

### (Insulators 33A and 33B)

As illustrated in FIGS. 2 to 6, the first insulator 33A forms the gas chamber 35, and the second insulator 33B forms the reference gas duct 36 and buries the heating element 34. The first insulator 33A and the second insulator 33B are made of a metallic oxide such as alumina (aluminum oxide). The insulators 33A and 33B are formed as dense bodies that is difficult for the detection target gas G or the reference gas A transit. The insulators 33A and 33B have few air holes through which a gas can pass.

### (Porous Layer 37)

As illustrated in FIG. 1, a porous layer 37 is provided on the entire circumference of a portion of the front end side L1 of the sensor element 2 in longitudinal direction L in order to catch substances poisonous to the pump electrode 311, condensed water in the exhaust pipe 7, and the like. The porous layer 37 is formed of porous ceramic (metallic oxide) such as alumina.

The porosity of the porous layer 37 is larger than the porosity of the diffusion resistance layer 32, and the rate of flow of the detection target gas G that can transit the porous layer 37 is higher than the rate of flow of the detection target gas G that can be transmitted through the diffusion resistance layer 32.

### (Another Configuration of the Gas Sensor 1)

As illustrated in FIG. 1, the gas sensor 1 includes, besides the sensor element 2, a first insulator 42 holding the sensor element 2, a housing 41 holding the first insulator 42, a second insulator 43 coupled to the first insulator 42, and the contact terminals 44 that are held by the second insulator 43 and in contact with the sensor element 2. The gas sensor 1 also includes: an element cover 45A, 45B that is attached to the portion of the housing 41 on the front end side L1 and cover the portion of the sensor element 2 on the front end side L1; a reference gas cover 46A, 46B that is attached to the portion of the housing 41 on the rear end side L2 and covers the second insulator 43, the contact terminals 44, and the like; and a bush 47 that holds the lead wires 48 connected to the contact terminals 44 in the reference gas cover 46A, 46B.

The portion of the sensor element 2 on the front end side L1 and the element cover 45A, 45B are arranged in the exhaust pipe 7 of the internal combustion engine. The element cover 45A, 45B have gas passage holes 451 for letting pass an exhaust gas as the detection target gas G The element cover 45A, 45B has a double structure which has the inner cover 45A and the outer cover 45B covering the inner cover 45A. The element cover 45A and 45B may be of a single structure. The detection target gas G flowing into the element cover 45A, 45B through the gas passage holes 451 of the element cover 45A, 45B passes through the porous layer 37 and the diffusion resistance layer 32 of the sensor element 2 and is guided into the gas chamber 35.

As illustrated in FIG. 1, the reference gas cover 46A, 46B is arranged outside the exhaust pipe 7 of the internal combustion engine. The reference gas cover 46A, 46B of the present embodiment includes the first cover 46A attached to the housing 41 and the second cover 46B covering the first cover 46A. The second cover 46B has an atmosphere passage hole 461 for letting the reference gas A pass therethrough. A water-repellent filter 462 is arranged inside the second cover 46B at a position facing the atmosphere passage hole 461 in order to prevent intrusion of water into the first cover 46A.

The rear end opening 361 of the reference gas duct 36 in the sensor element 2 is opened to the space within the reference gas cover 46A, 46B. The reference gas A existing around the atmosphere passage hole 461 of the reference gas cover 46A, 46B is taken into the reference gas cover 46A, 46B through the water-repellent filter 462. Then, the reference gas A having passed through the water-repellent filter 462 flows into the reference gas duct 36 through the rear end opening 361 of the reference gas duct 36 in the sensor element 2 and is guided into the reference electrode 314 in the reference gas duct 36.

The plurality of contact terminals 44 are arranged in the second insulator 43 and connected to the respective electrode lead portions 311X, 312X, 314X, and 314Y of the pump electrode 311, the sensor electrode 312, the pump-side electrode portion 314A and sensor-side electrode portion 314B of the reference electrode 314, and the heating element lead portions 342 of the heating element 34, respectively. The lead wires 48 are connected to the corresponding contact terminals 44.

### (Sensor Control Unit 6)

As illustrated in FIGS. 1 and 2, the lead wires 48 in the gas sensor 1 are electrically connected to the sensor control unit (SCU) 6 that controls gas detection in the gas sensor 1. The sensor control unit 6 performs electrical control of the gas sensor 1 in cooperation with an engine control unit (ECU) that controls combustion operation in the engine.

The sensor control unit 6 includes a pump voltage application circuit 61, a sensor voltage application circuit 62, a sensor current detection circuit 64, an energization circuit, and the like. The pump voltage application circuit 61 applies a direct-current voltage between the pump electrode 311 and the pump-side electrode portion 314A of the reference electrode 314. The sensor voltage application circuit 62 applies a direct-current voltage between the sensor electrode 312 and the sensor-side electrode portion 314B of the reference electrode 314. The sensor current detection circuit 64 measures the electric current flowing between the sensor electrode 312 and the sensor-side electrode portion 314B of the reference electrode 314. The energization circuit energizes the heating element 34. The sensor control unit 6 may be provided in the engine control unit.

When the pump voltage application circuit 61 applies a direct-current voltage between the pump electrode 311 and the pump-side electrode portion 314A of the reference electrode 314 such that the pump-side electrode portion 314A is on the positive side, the oxygen contained in the detection target gas G is discharged from the pump electrode 311 into the pump-side electrode portion 314A via the solid electrolyte body 31. Accordingly, the concentration of oxygen in the detection target gas G within the gas chamber 35 is maintained at a predetermined concentration or less.

When the sensor voltage application circuit 62 applies a direct-current voltage between the sensor electrode 312 and the sensor-side electrode portion 314B of the reference electrode 314 such that the sensor-side electrode portion 314B is on the positive side, the diffusion resistance layer 32 causes diffusion control to limit the electric current. In this state, if the concentration of NOx as the specific gas component changes, the electric current in accordance with the amount of NOx decomposed in the sensor electrode 312 flows between the sensor electrode 312 and the sensor-side electrode portion 314B. The current flowing between the sensor electrode 312 and the sensor-side electrode portion 314B is detected by the sensor current detection circuit 64.

### (Method for Manufacturing the Sensor Element 2)

To manufacture the sensor element 2, a paste material to constitute the pump electrode 311, the sensor electrode 312, and the reference electrode 314 is printed (applied) on a sheet to constitute the solid electrolyte body 31, and a paste material to constitute the heating element 34 is printed (applied) on a sheet to constitute the second insulator 33B. In addition, a paste material to constitute the shield layer 5 is printed (applied) on the surface of the paste material to constitute the sensor-side electrode portion 314B of the reference electrode 314. Then, the sheet to constitute the solid electrolyte body 31, the sheet to constitute the first insulator 33A, and the sheet to constitute the second insulator 33B are laminated and adhered to one another via adhesive layers. After that, the intermediate to constitute the sensor element 2 formed by the above sheets and paste materials is fired at a predetermined firing temperature to form the sensor element 2.

### (Detection Operation and Operational Effects of the Gas Sensor 1)

At the time of use of the gas sensor 1, when the operational state of the internal combustion engine becomes the fuel-rich state or the fuel-lean state, the sensor current detection circuit 64 of the gas sensor 1 detects the concentration of NOx in the detection target gas G In the fuel-lean state, in particular, the concentration of NOx in the detection target gas G increases. In the fuel-rich state or the fuel-lean state, the oxygen in the air (atmosphere) taken into the internal combustion engine is used for combustion and thus the concentration of oxygen in the exhaust gas as the detection target gas G is not so high.

On the other hand, when the operational state of the internal combustion engine enters the fuel-cut state in which the injection of the fuel is stopped, the oxygen in the air taken into the internal combustion engine is not used for combustion and thus the concentration of oxygen in the exhaust gas as the detection target gas G exhausted from the internal combustion engine sharply increases to the degree equal to the concentration of oxygen in the air. In the gas sensor 1, a large amount of oxygen is pumped from the pump electrode 311 into the pump-side electrode portion 314A via the solid electrolyte body 31 due to the direct-current voltage applied between the pump electrode 311 and the pump-side electrode portion 314A of the reference electrode 314. At this time, the concentration of oxygen in the reference gas A within the reference gas duct 36 increases temporarily.

In the gas sensor 1 of the present embodiment, the sensor-side electrode portion 314B of the reference electrode 314 is covered with the shield layer 5 and is shielded by the shield layer 5 from the atmosphere as the reference gas A within the reference gas duct 36. Thus, even if the concentration of oxygen in the reference gas A within the reference gas duct 36 increases temporarily, the concentration of oxygen in the reference gas A in contact with the sensor-side electrode portion 314B covered with the shield layer 5 hardly changes.

Accordingly, when the sensor current detection circuit 64 detects the electric current flowing between the sensor electrode 312 and the sensor-side electrode portion 314B, the electric current is hardly affected by the temporary increase of the concentration of oxygen in the reference gas A within the reference gas duct 36. As a result, it is possible to maintain a high degree of accuracy of detecting the concentration of NOx as a specific gas component, in accordance with the electric current flowing between the sensor electrode 312 and the sensor-side electrode portion 314B.

Therefore, according to the gas sensor 1 of the present embodiment, even if the concentration of oxygen in the reference gas A sharply increases, it is possible that there is almost no error in the detection of concentration of the specific gas component.

The shield layer 5 may also serve as a poisoning prevention layer that prevents substances possibly poisonous to the reference electrode 314 from attaching to the sensor-side electrode portion 314B of the reference electrode 314. The poisonous substances include silicon (Si) contained in the atmosphere, which could be generated in the engine compartment where the internal combustion engine is arranged and flow from the engine compartment into the reference gas cover of the gas sensor 1.

### (Another Shield Layer 5)

The shield layer 5 may be formed of a porous ceramic material with the property of allowing reference gas to transit A. The “porous” means that there are gaps between particles of a metallic oxide constituting the ceramic material to a degree that a gas such as the reference gas A can pass through.

In this case, the reference gas A in the reference gas duct 36 can be allowed to reach the sensor-side electrode portion 314B of the reference electrode 314 covered with the shield layer 5. On the other hand, when the concentration of oxygen in the reference gas A within the reference gas duct 36 increases temporarily, the presence of the shield layer 5 makes the reference gas A having temporarily increased in oxygen concentration unlikely to contact the sensor-side electrode portion 314B. Accordingly, it is possible to suppress the occurrence of an error in the detection of the concentration of NOx as the specific gas component while maintaining the ease with which the reference gas A reaches the sensor-side electrode portion 314B.

### Second Embodiment

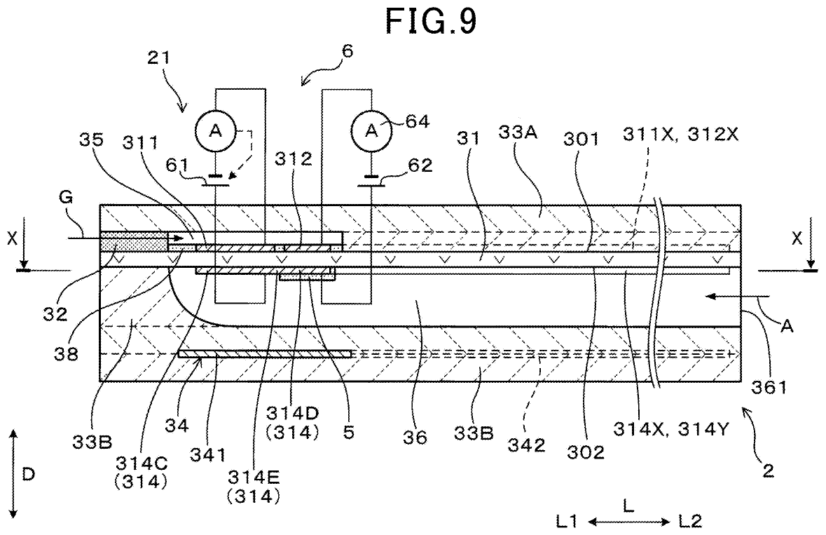

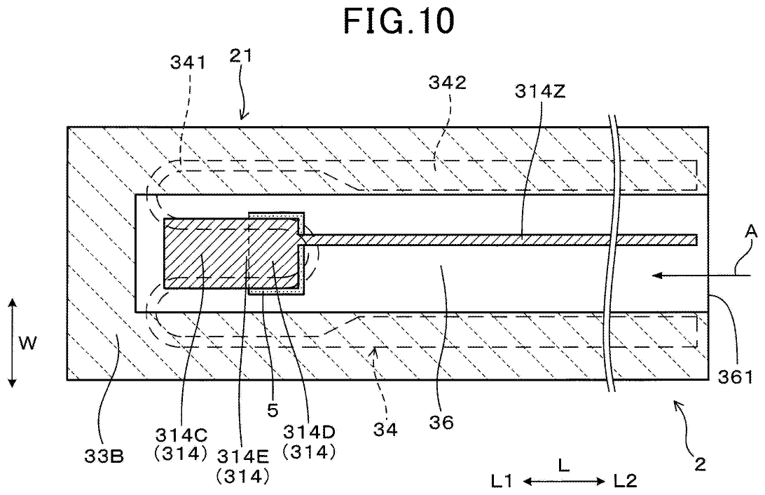

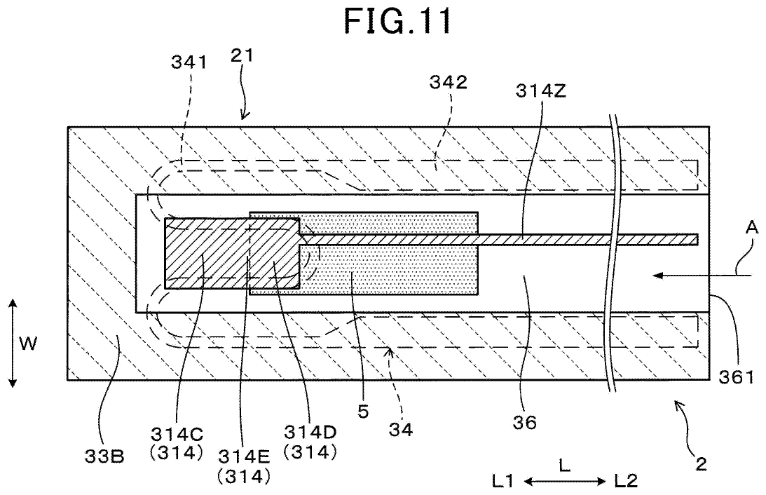

As illustrated in FIGS. 9 and 10, a reference electrode 314 in a sensor element 2 of a gas sensor 1 of the present embodiment includes a pump-side portion 314C arranged to overlap a pump electrode 311 in a lamination direction D through a solid electrolyte body 31 and a sensor-side portion 314D arranged to integrally extend from the pump-side portion 314C to overlap a sensor electrode 312 in the lamination direction D through the solid electrolyte body 31. In other words, the reference electrode 314 of the present embodiment is continuously formed at a position where to overlap the pump electrode 311 and the sensor electrode 312 in the lamination direction D through the solid electrolyte body 31. A connect portion 314E is positioned between the pump-side portion 314C and the sensor-side portion 314D of the reference electrode 314, which overlaps the gap between the pump electrode 311 and the sensor electrode 312 in the lamination direction D through the solid electrolyte body 31.

A reference electrode lead portion 314Z connected to the reference electrode 314 is formed from the rear end portion of the reference electrode 314 along the longitudinal direction L to the rear end portion of the solid electrolyte body 31 along the longitudinal direction L. In order to facilitate the intake of the oxygen of the reference gas A into the reference electrode lead portion 314Z, the area of the cross section of the reference electrode lead portion 314Z orthogonal to the longitudinal direction L can be made larger than the area of the cross section of a pump electrode lead portion 311X orthogonal to the longitudinal direction L and the area of the cross section of a sensor electrode lead portion 312X orthogonal to the longitudinal direction L. The area of the cross section orthogonal to the longitudinal direction L can be altered by changing at least one of the thickness along the lamination direction D and the width along the width direction W.

The shield layer 5 in the present embodiment is provided so as not to bury the flow path in the reference gas duct 36 but so as to contact the sensor-side portion 314D of the reference electrode 314 and bury the sensor-side portion 314D. In other words, the shield layer 5 is not provided on the surface of the pump-side portion 314C but is provided only on the surface of the sensor-side portion 314D. The shield layer 5 may be or may not be provided on a connect portion 314E.

The shield layer 5 is provided on a second surface 302 of the solid electrolyte body 31, in contact with the both sides of the sensor-side portion 314D along the width direction W and a portion of the sensor-side portion 314D on the rear end side L2 in the longitudinal direction L. The shield layer 5 may be formed of either a dense ceramic material or a porous ceramic material. As illustrated in FIG. 11, the shield layer 5 may continuously cover an entire sensor-side electrode portion 314B and a portion of the reference electrode lead portion 314Z.

Other configurations and operational effects of the gas sensor 1 of the present embodiment are the same as those of the first embodiment. The components of the gas sensor 1 of the present embodiment denoted by reference signs identical to those of the first embodiment are the same as the components of the first embodiment.

### Third Embodiment

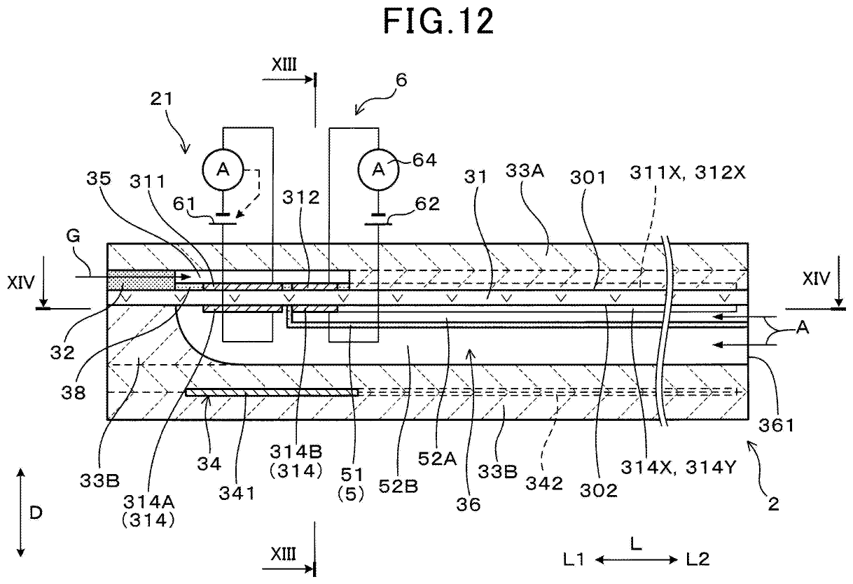

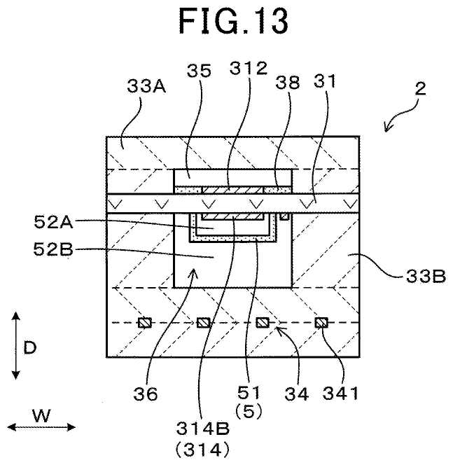

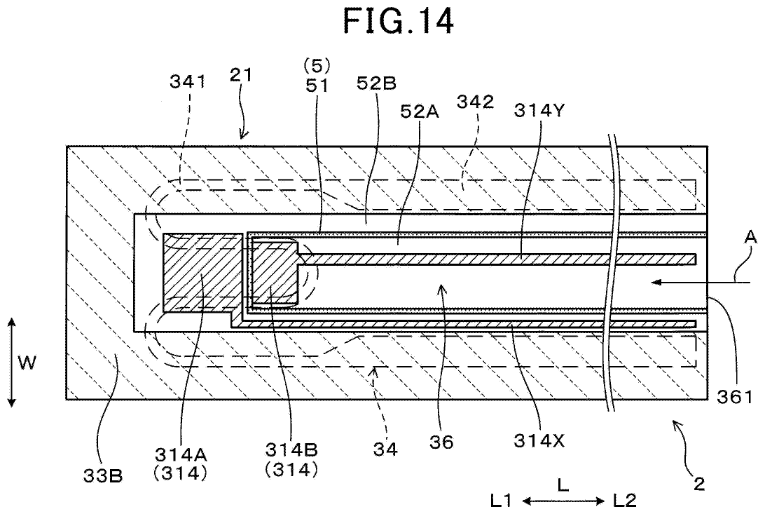

As illustrated in FIGS. 12 to 14, a shield layer 5 in a sensor element 2 of a gas sensor 1 of the present embodiment and a second surface 302 of a solid electrolyte body 31 are arranged to surround the sensor-side electrode portion 314B of a reference electrode 314 within the reference gas duct 36 to define an internal duct 51 between the shield layer 5 and the second surface 302 of the solid electrolyte body 31, each of the reference gas duct 36 and the internal duct 51 having a flow path of the reference gas, the flow path of the reference gas duct being separated from the flow path of the internal duct. The internal duct 51 covers the sensor-side electrode portion 314B in a non-contact state with the sensor-side electrode portion 314B. The internal duct 51 is formed to divide the flow path of the reference gas duct 36 into an inner flow path 52A inside the internal duct 51 and an outer flow path 52B outside the internal duct 51.

The internal duct 51 is formed in a tubular by using the second surface 302 of the solid electrolyte body 31. The inner flow path 52A and the outer flow path 52B are formed from the rear end portion of the reference gas duct 36 along the longitudinal direction L to the position of the sensor-side electrode portion 314B. The front end portion of the internal duct 51 along the longitudinal direction L is closed between a pump-side electrode portion 314A and the sensor-side electrode portion 314B. Through a rear end opening 361 of the reference gas duct 36, a reference gas A flows in the internal duct 51, divided into the inner flow path 52A and the outer flow path 52B.

The internal duct 51 is made of a dense ceramic material with the property that is difficult for reference gas to transit A. The internal duct 51 can be formed by various methods. The internal duct 51 can be formed, for example, by the method described below. A paste of resin burning agent, which will burn off at the time of firing the sensor element 2, is applied to the sensor-side electrode portion 314B of the reference electrode 314 and to the second surface 302 of the solid electrolyte body 31, and a paste of ceramic material is applied to the surface of the burning agent paste. Then, when the sensor element 2 is fired, the burning agent paste burns off, to form a cavity serving as the inner flow path 52A in the burned portion of the paste, and the ceramic material paste forms the inner duct 51.

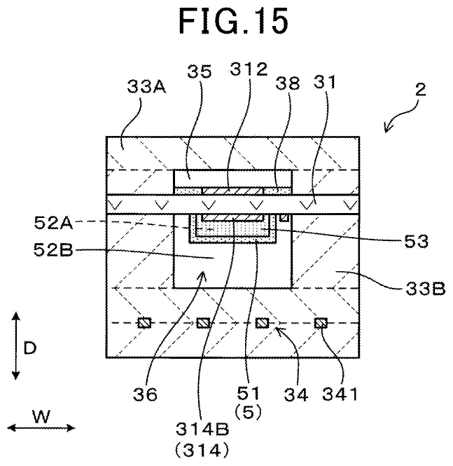

As illustrated in FIG. 15, the internal duct 51 can be filled with a porous ceramic material with the property of allowing reference gas to transit A In other words, a gas transmission layer 53 of porous ceramic material can be formed inside the internal duct 51. Filling the internal duct 51 with the porous ceramic material (forming the gas transmission layer 53) makes the space in the internal duct 51 less likely to be damaged during firing of the sensor element 2.

The dense ceramic material and the porous ceramic material are both made of particles of a metallic oxide such as alumina. The dense ceramic material has the property that is difficult for reference gas A to transit because there are few gaps between particles of the metallic oxide. The porous ceramic material has the property of allowing the reference gas A to transit because the gaps between particles of the metallic oxide are larger than those of the dense ceramic material.

As illustrated in FIGS. 12 to 14, a second reference electrode lead portion 314Y connected to the rear end position of the sensor-side electrode portion 314B of the reference electrode 314 along the longitudinal direction L is arranged in the inner flow path 52A inside the internal duct 51. A first reference electrode lead portion 314X connected to the rear end position of the pump-side electrode portion 314A of the reference electrode 314 along the longitudinal direction L is arranged in the outer flow path 52B outside the internal duct 51.

In the present embodiment, the internal duct 51 can separate the reference gas A to contact the sensor-side electrode portion 314B of the reference electrode 314 from the reference gas A to contact the pump-side electrode portion 314A of the reference electrode 314.

Accordingly, even if the concentration of oxygen in the reference gas A in the outer flow path 52B in which the pump-side electrode portion 314A is arranged increases temporarily due to the fuel-cut state of the internal combustion engine, the concentration of oxygen in the reference gas A in the inner flow path 52A in which the sensor-side electrode portion 314B is arranged can remain unchanged.

The use of the internal duct 51 can also provide a sufficient supply of the reference gas A unaffected by changes in the potential of the pump-side electrode portion 314A from the inner flow path 52A to the sensor-side electrode portion 314B. Then, it is possible to make it difficult for an error to occur in the detection of the concentration of NOx as the specific gas component, while maintaining the ease with which the reference gas A reaches the sensor-side electrode portion 314B.

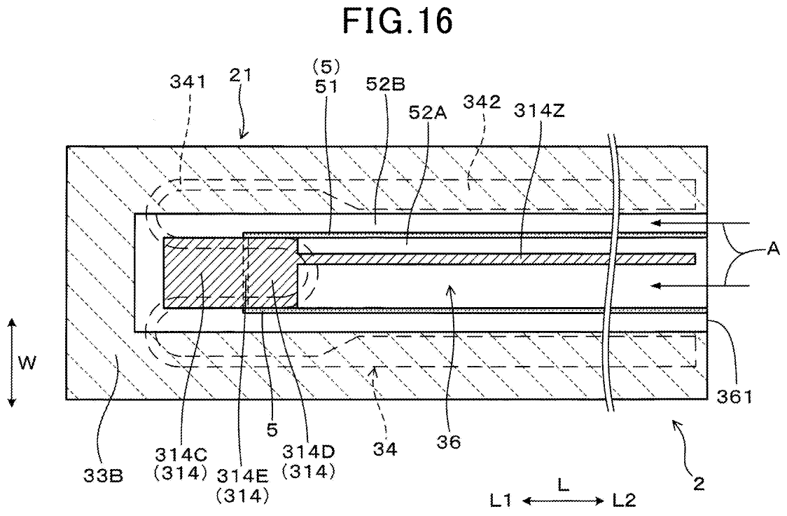

As illustrated in FIG. 16, in the reference electrode 314 of the present embodiment, the pump-side portion 314C may be arranged to integrally extend from the sensor-side portion 314D as in the second embodiment. In this case, the internal duct 51 can be formed to surround the sensor-side portion 314D of the reference electrode 314 and divide the flow path of the reference gas duct 36 into the inner flow path 52A inside the internal duct 51 and the outer flow path 52B outside the internal duct 51.

Other configurations, operational effects, and the like of the gas sensor 1 of the present embodiment are the same as those of the first and second embodiments. The components of the gas sensor 1 of the present embodiment denoted by reference signs identical to those of the first and second embodiments are the same as those of the first and second embodiments.

### Fourth Embodiment

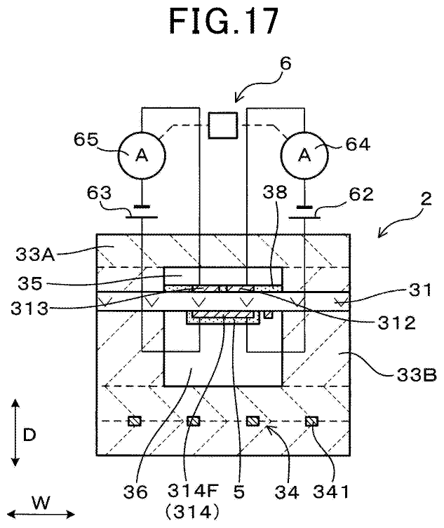

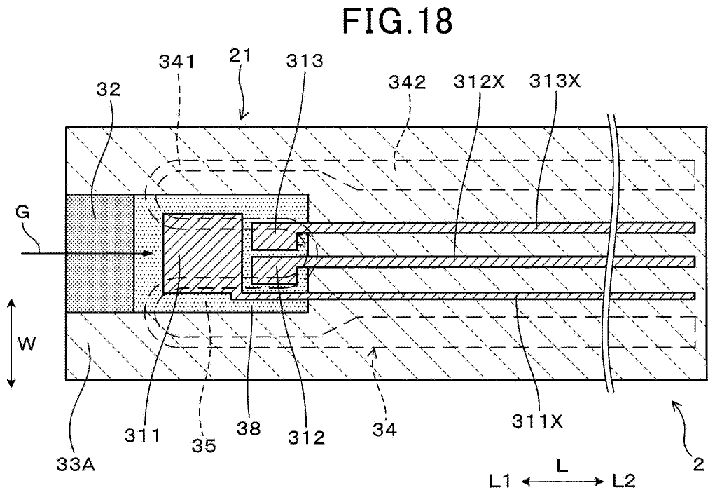

As illustrated in FIGS. 17 and 18, a sensor element 2 of a gas sensor 1 of the present embodiment has, besides a pump electrode 311, a sensor electrode 312, and a reference electrode 314, a monitor electrode 313 for detecting the concentration of oxygen in a detection target gas G that includes the oxygen whose concentration has been adjusted by the pump electrode 311.

The monitor electrode 313 is provided on a first surface 301 of a solid electrolyte body 31 at a position adjacent to a rear end side L2 of the pump electrode 311 along a longitudinal direction L and adjacent to the sensor electrode 312 along a width direction W.

The pump electrode 311, the sensor electrode 312, the monitor electrode 313, the reference electrode 314, and a gas chamber 35 are provided at portions of the sensor element 2 on a front end side L1 in the longitudinal direction L. The reference electrode 314 of the present embodiment is provided to overlap the pump electrode 311 in a lamination direction D through the solid electrolyte body 31 and to cover the sensor electrode 312 and the monitor electrode 313 in the lamination direction D through the solid electrolyte body 31. More specifically, the reference electrode 314 includes a pump-side electrode portion 314A and a sensor/monitor-side electrode portion 314F. The pump-side electrode portion 314A is provided to cover the pump electrode 311 in the lamination direction D through the solid electrolyte body 31, and the sensor/monitor-side electrode portion 314F is provided to cover the sensor electrode 312 and the monitor electrode 313 in the lamination direction D through the solid electrolyte body 31. The pump-side electrode portion 314A and the sensor/monitor-side electrode portion 314F are separated from each other. The sensor/monitor-side electrode portion 314F includes a connect portion 314G overlapping the gap between the sensor electrode 312 and the monitor electrode 313 in the lamination direction D.

A monitor electrode lead portion 313X provided on the first surface 301 of the solid electrolyte body 31 is connected to the rear end position of the monitor electrode 313 along the longitudinal direction L. The monitor electrode lead portion 313X is formed from the rear end position of the monitor electrode 313 along the longitudinal direction L to the rear end portion of the first surface 301 of the solid electrolyte body 31 along the longitudinal direction L.

A second reference electrode lead portion 314Y provided on a second surface 302 of the solid electrolyte body 31 is connected to the rear end position of the sensor/monitor-side electrode portion 314F of the reference electrode 314 along the longitudinal direction L. The second reference electrode lead portion 314Y is formed from the rear end position of the sensor/monitor-side electrode portion 314F along the longitudinal direction L to the rear end portion of the second surface 302 of the solid electrolyte body 31 along the longitudinal direction L. The pump electrode lead portion 311X, the sensor electrode lead portion 312X, and the first reference electrode lead portion 314X are formed in the same manner as those of the first embodiment.

As illustrated in FIG. 17, a sensor control unit 6 of the present embodiment includes a monitor voltage application circuit 63 that applies a direct-current voltage between the monitor electrode 313 and the sensor/monitor-side electrode portion 314F such that the sensor/monitor-side electrode portion 314F is on the positive side, and a monitor current detection circuit 65 that measures the electric current flowing between the monitor electrode 313 and the sensor/monitor-side electrode portion 314F. The sensor control unit 6 can also subtract the value of the electric current detected by the monitor current detection circuit 65 of the monitor electrode 313 from the value of the electric current detected by the sensor current detection circuit 64 of the sensor electrode 312, thereby to alleviate the influence of the residual oxygen remaining in the gas chamber 35 after the pumping of the oxygen from the gas chamber 35 by the pump electrode 311 on the detection of the concentration of NOx as a specific gas component. The configurations of the sensor voltage application circuit 62 and the sensor current detection circuit 64 are the same as those of the first embodiment.

A shield layer 5 of the present embodiment is formed to cover the entire sensor/monitor-side electrode portion 314F. The shield layer 5 may cover continuously the entire sensor/monitor-side electrode portion 314F and at least a portion of the second reference electrode lead portion 314Y along the longitudinal direction L.

The reference electrode 314 may be arranged on the second surface 302 of the solid electrolyte body 31, at a first position overlapping the pump electrode 311 in the lamination direction D, a second position overlapping the sensor electrode 312 in the lamination direction D, and a third position overlapping the monitor electrode 313 in the lamination direction D, the first position, the second position, and the third position are separated from each other.

The reference electrode 314 may be integrally provided to cover the pump electrode 311, the sensor electrode 312, and the monitor electrode 313 in the lamination direction D through the solid electrolyte body 31. In this case, the shield layer 5 can be provided to cover a portion of the reference electrode 314 overlapping the sensor electrode 312 and the monitor electrode 313 in the lamination direction D through the solid electrolyte body 31.

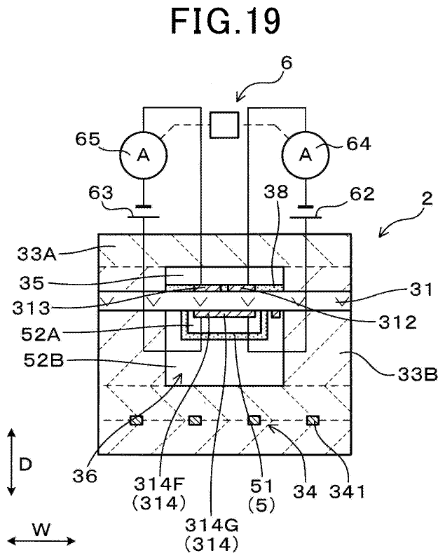

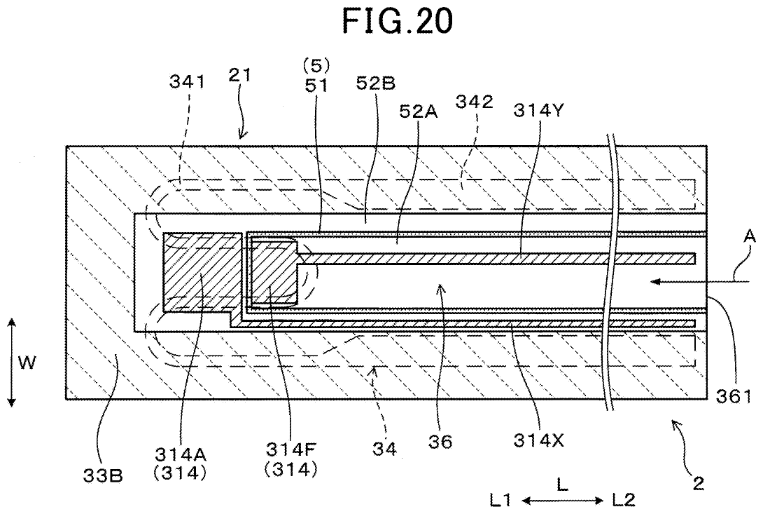

The shield layer 5 may be made of a dense ceramic material or may be formed of a porous ceramic material, as illustrated in relation to the first embodiment. As illustrated in FIGS. 19 and 20, the shield layer 5 may be made of the internal duct 51 illustrated in relation to the third embodiment. In this case, the sensor/monitor-side electrode portion 314F of the reference electrode 314 or the portion of the reference electrode 314 facing the sensor electrode 312 and the monitor electrode 313 is arranged in an inner flow path 52A of the internal duct 51.

Other configurations, operational effects, and the like of the gas sensor 1 of the present embodiment are the same as those of the first to third embodiments. The components of the gas sensor 1 of the present embodiment denoted by reference signs identical to those of the first to third embodiments are the same as those of the first to third embodiments.

### <Verification Test>

In this verification test, samples of the gas sensor 1 (test samples 1 to 4) were arranged in an evaluation pipe simulating the exhaust pipe 7 of an internal combustion engine, and the detection target gas G was flown into the evaluation pipe with different concentrations of oxygen, and then the changes in the output of a sensor for detecting NOx as a specific gas component in the detection target gas G were measured. A heater for heating the detection target gas G was arranged on the outer periphery of the evaluation pipe to heat the detection target gas G to 25° C.

The flow rate of the detection target gas G in the evaluation pipe was set to 3 m/s. The detection target gas G was changed from a state with an oxygen concentration of 0 volume % to a state with an oxygen concentration of 20 volume %. The state with an oxygen concentration of 0 volume % simulated the stoichiometric state of an internal combustion engine, where only nitrogen (N₂) would flow. The state with an oxygen concentration of 20 volume % simulated the fuel-cut state of an internal combustion engine, where nitrogen (N₂) and oxygen (O₂) would flow.

As the samples of the gas sensor 1, the test samples 1 to 4 different in the formation of the shield layer 5 and a comparative sample without the shield layer 5 were employed. The test sample 1 is the gas sensor 1 of the first embodiment in which the sensor-side electrode portion 314B of the reference electrode 314 is covered with the shield layer 5. The test sample 2 is the gas sensor 1 of the third embodiment in which the sensor-side electrode portion 314B of the reference electrode 314 is arranged in the internal duct 51 as the shield layer 5. The test sample 3 is the gas sensor 1 of the third embodiment in which a porous ceramic material is filled in the internal duct 51. The test sample 4 is the gas sensor 1 of the second embodiment in which the sensor-side portion 314D of the reference electrode 314 is covered with the shield layer 5.

The concentration of oxygen in the detection target gas G to contact the sensor element 2 of each of the test samples 1 to 4 and the comparative sample was changed from 0 volume % to 20 volume %, and then it was verified how much the electric current flowing between the sensor electrode 312 and the reference electrode 314 changed, as a sensor output. For evaluation of the sensor output, amounts of decrease in the sensor output of ±10% or less were rated as excellent and indicated with “excellent”, and amounts of decrease in the sensor output of ±30% were rated as good and indicated with “good”. In addition, an amount of decrease in the sensor output of more than ±30% was rated as poor and indicated with “poor”.

The evaluation results of the verification test are shown in Table 1.

TABLE 1

| | | Presence or absence | Judgment |
|---|---|---|---|
| | | of sensor output | of sensor |
| | Structure | fluctuation | output |
| Test | First embodiment (shield | Hardly fluctuated | excellent |
| sample 1 | layer on separated | | |
| | reference electrode) | | |
| Test | Third embodiment (shield | Hardly fluctuated | excellent |
| sample 2 | layer as internal duct) | | |
| Test | Third embodiment (porous | Slightly fluctuated | good |
| sample 3 | material in internal duct) | | |
| Test | Second embodiment (shield | Slightly fluctuated | good |
| sample 4 | layer on integrated | | |
| | reference electrode) | | |
| Comparative | No shield layer | Fluctuated | poor |
| example | | | |

Referring to the above table, it has been revealed that the sensor outputs of the test samples 1 and 2 were rated as “excellent” and that high detection accuracies can be obtained with minimum error in sensor output according to the gas sensors 1 of the first and third embodiments. It has been found that the sensor outputs of the test samples 3 and 4 were rated as “good” and that errors in sensor output can be suppressed to a small value according to the gas sensors of the second embodiment and another example of the third embodiment. On the other hand, it has been discovered that the sensor output of the comparative test sample was rated “poor” and that an error in sensor output would increase and the output accuracy would temporarily decrease without the shield layer 5 in the sensor element 2.

From the above verification test results, it has been verified that the gas sensors 1 in the first to fourth embodiments maintain high degrees of accuracy of detecting the concentration of the specific gas component.

The present disclosure is not limited to the above embodiments but can constitute further different embodiments without departing from the gist of the present disclosure. The present disclosure includes various modifications and modifications within the equivalent range. Further, various combinations and modes of components conceived from the present disclosure are included in the technical idea of the present disclosure.

What is claimed is:

1. A gas sensor that includes a sensor element for detecting the concentration of a specific gas component in a detection target gas, wherein
the sensor element includes:
a solid electrolyte body that has ionic conductivity, and has a first surface and a second surface;
a first insulator that is laminated on the first surface of the solid electrolyte body and has a recess;
a gas chamber into which the detection target gas is introduced, the gas chamber being defined by the first surface of the solid electrolyte body and the recess of the first insulator;
a second insulator that is laminated on the second surface of the solid electrolyte body, and has a groove;
a reference gas duct into which a reference gas is introduced, the reference gas duct being defined by the second surface of the solid electrolyte body and the groove of the second insulator;
a pump electrode for adjusting the concentration of oxygen in the detection target gas, the pump electrode being provided on the first surface of the solid electrolyte body and housed in the gas chamber;
a sensor electrode for detecting the concentration of the specific gas component in the detection target gas that includes the oxygen whose concentration has been adjusted by the pump electrode, the sensor electrode being provided on the first surface of the solid electrolyte body and housed in the gas chamber;
a reference electrode arranged on the second surface of the solid electrode to overlap both the pump electrode and the sensor electrode through the solid electrolyte body; and
an insulating shield layer arranged to cover a portion of the reference electrode in a contact state or non-contact state with the portion of the reference electrode, the portion of the reference electrode being arranged to overlap the sensor electrode through the solid electrolyte body.

2. The gas sensor according to claim 1, wherein
the portion of the reference electrode is a sensor-side electrode portion arranged to overlap the sensor electrode through the solid electrolyte body;
the reference electrode further includes a pump-side electrode arranged to overlap the pump electrode through the solid electrolyte body, the pump-side electrode portion and the sensor-side electrode portion being separated from each other, and
the shield layer and the second surface of the solid electrolyte body are arranged to surround the sensor-side electrode portion within the reference gas duct to define an internal duct between the shield layer and the second surface of the solid electrolyte body, each of the reference gas duct and the internal duct having a flow path of the reference gas, the flow path of the reference gas duct being separated from the flow path of the internal duct.

3. The gas sensor according to claim 1, wherein
the portion of the reference electrode is a first electrode portion arranged to overlap the sensor electrode through the solid electrolyte body;
the reference electrode further includes a second electrode portion arranged to integrally extend from the first electrode portion to overlap the pump electrode through the solid electrolyte body, and
the shield layer and the second surface of the solid electrolyte body are arranged to surround the first electrode portion within the reference gas duct to define an internal duct between the shield layer and the second surface of the solid electrolyte body, each of the reference gas duct and the internal duct having a flow path of the reference gas, the flow path of the reference gas duct being separated from the flow path of the internal duct.

4. The gas sensor according to claim 1, wherein
the portion of the reference electrode is a sensor-side electrode portion arranged to overlap the sensor electrode through the solid electrolyte body;
the reference electrode further includes a pump-side electrode arranged to overlap the pump electrode through the solid electrolyte body, the pump-side electrode portion and the sensor-side electrode portion being separated from each other, and
the shield layer is provided so as not to bury a flow path in the reference gas duct but so as to be in contact with the sensor-side electrode portion and bury the sensor-side electrode portion.

5. The gas sensor according to claim 1, wherein
the portion of the reference electrode is a first electrode portion arranged to overlap the sensor electrode through the solid electrolyte body;
the reference electrode further includes a second electrode portion arranged to integrally extend from the first electrode portion to overlap the pump electrode through the solid electrolyte body, and
the shield layer is provided so as not to bury a flow path in the reference gas duct but so as to be in contact with the first electrode portion and bury the first electrode portion.

6. The gas sensor according to claim 2, wherein the shield layer is formed of a dense ceramic material with the property that is difficult for reference gas to transit.

7. The gas sensor according to claim 2, wherein
the internal duct is formed of a dense ceramic material with the property that is difficult for reference gas to transit, and
the internal duct is filled with a porous ceramic material with the property of allowing reference gas to transit.

8. The gas sensor according to claim 4, wherein the shield layer is formed of a porous ceramic material with the property of allowing reference gas to transit.

9. The gas sensor according to claim 1, wherein
the reference electrode is connected to a reference electrode lead portion for external connection that is provided on the second surface of the solid electrolyte body, and
the shield layer continuously covers the portion of the reference electrode being arranged to overlap the sensor electrode through the solid electrolyte body and at least part of the reference electrode lead portion.

10. The gas sensor according to claim 9, wherein
the sensor element has a rectangular parallelepiped shape,
the pump electrode is connected to a pump electrode lead portion for external connection that is provided on the first surface of the solid electrolyte body,
the sensor electrode is connected to a sensor electrode lead portion for external connection that is provided on the first surface of the solid electrolyte body,
the reference electrode is connected to a reference electrode lead portion for external connection that is provided on the second surface of the solid electrolyte body, and
the pump electrode lead portion, the sensor electrode lead portion, and the reference electrode lead portion extend to a rear end portion of the sensor element along a longitudinal direction, and
the area of a cross section of the reference electrode lead portion orthogonal to the longitudinal direction is larger than the area of a cross section of the pump electrode lead portion orthogonal to the longitudinal direction and the area of a cross section of the sensor electrode lead portion orthogonal to the longitudinal direction.

11. The gas sensor according to claim 1, wherein
the sensor element has a rectangular parallelepiped shape,
the pump electrode, the sensor electrode, the reference electrode, and the gas chamber are formed at a portion on a front end side of the sensor element in the longitudinal direction,
a diffusion resistance layer is provided on a front end portion of the first insulator in the longitudinal direction to introduce the detection target gas under a predetermined diffusion resistance,
a monitor electrode for detecting the concentration of oxygen in the detection target gas after adjustment of the concentration of oxygen by the pump electrode is provided on the first surface of the solid electrolyte body and provided at a position adjacent to the sensor electrode, and
the reference electrode is arranged to cover the pump electrode, the sensor electrode, and the monitor electrode through the solid electrolyte body, and
the shield layer is arranged to cover a portion of the reference electrode, the portion of the reference electrode being arranged to overlap both the sensor electrode and the monitor electrode through the solid electrolyte body.
